(19) United States
(12) Patent Application Publication
(10) Pub. No.: US 2022/0133314 A1
(43) Pub. Date: May 5, 2022

(54) SURGICAL STAPLING DEVICE WITH PIVOTABLE KNIFE BLADE

(71) Applicant: Covidien LP, Mansfield, MA (US)

(72) Inventor: Shouwei Li, Shanghai (CN)

(21) Appl. No.: 17/437,585

(22) PCT Filed: Apr. 9, 2019
(86) PCT No.: PCT/CN2019/081939

§ 371 Date: Sep. 9, 2021

(51) Int. Cl. A61B 17/072

(52) U.S. Cl. CPC A61B 17/072; A61B 2017/07285; A61B 2017/07278; A61B 2017/07271; A61B 2017/07257

(57) ABSTRACT

Disclosed is a surgical stapling device (10). It includes a tool assembly (16) including a cartridge assembly (30a), an anvil assembly (32a), and a drive assembly (34). The drive assembly (34) includes a working member (52) and a first knife (74) that is pivotally supported on the working member (52) and has a first cutting edge (74a). The first knife (74) is positioned to extend between the cartridge and anvil assemblies (30a, 32a) such that the first cutting edge (74a) extends across a tissue gap of the tool assembly (16) when the tool assembly (16) is in the clamped position.

### FIELD

The disclosure is directed to surgical stapling devices and, more particularly, to a surgical stapling device including a tool assembly with a knife blade that can pivot in relation to tissue.

### BACKGROUND

Surgical stapling devices for simultaneously cutting and stapling tissue are known in the art and are commonly used during surgical procedures to reduce the time required to perform the surgical procedure and to facilitate endoscopic access to a surgical site. Performing a surgical procedure endoscopically reduces the amount of trauma inflicted on a patient during a surgical procedure to minimize patient discomfort and reduce patient recovery times.

Typically, endoscopic stapling devices include a tool assembly having a first jaw and a second jaw that can pivot in relation to the first jaw between an open or spaced position and a closed or clamped position. One of the jaws supports a cartridge assembly that includes a plurality of staples and the other jaw supports an anvil assembly that includes an anvil plate that includes staple deforming pockets that receive and deform legs of the staples when the staples are ejected from the staple cartridge.

In known devices, each of the first and second jaws defines a knife slot that receives a knife blade that extends between the jaws of the tool assembly. The knife blade is configured to move through tool assembly to cut tissue clamped between the jaws as the staples are formed in the tissue. Typically, the direction of movement on the knife blade applies a horizontal force on the tissue which may lead to tissue accumulation and slippage during staple formation. This may have a negative impact on staple formation.

### SUMMARY

One aspect of the disclosure is directed to a tool assembly including a cartridge assembly, an anvil assembly, and a drive assembly. The cartridge assembly includes a staple cartridge that supports a plurality of staples and defines a first knife slot. The anvil assembly is coupled to the cartridge assembly by a pivot member to facilitate movement of the tool assembly between open and clamped positions. The anvil assembly defines a second knife slot that is aligned with the first knife slot of the cartridge assembly in the clamped position of the tool assembly. The tool assembly defines a tissue gap between the cartridge assembly and the anvil assembly when the tool assembly is in the clamped position. The drive assembly includes a drive beam having proximal and distal ends and a working member supported on the distal end of the drive beam. The working member includes an upper beam, a lower beam, and a vertical strut interconnecting the upper beam and the lower beam. The working member supports a first knife having a first or distal cutting edge supported on the vertical strut by a pivot member. The first knife is positioned to extend between the cartridge and anvil assemblies such that the distal cutting edge extends across the tissue gap of the tool assembly when the tool assembly is in the clamped position.

In another aspect of the disclosure, a tool assembly includes a cartridge assembly, an anvil assembly, and a drive assembly. The cartridge assembly includes a staple cartridge supporting a plurality of staples and defining a first knife slot. The anvil assembly is coupled to the cartridge assembly by a pivot member to facilitate movement of the tool assembly between open and clamped positions. The anvil assembly defines a second knife slot that is aligned with the first knife slot of the cartridge assembly in the clamped position. The tool assembly defines a tissue gap between the cartridge assembly and the anvil assembly when the tool assembly is in the clamped position. The drive assembly includes a working member and a first knife that is pivotally supported on the working member and has a first cutting edge. The first knife is positioned to extend between the cartridge and anvil assemblies such that the first cutting edge extends across the tissue gap of the tool assembly when the tool assembly is in the clamped position.

In another aspect of the disclosure, a tool assembly includes a cartridge assembly, an anvil assembly and a drive assembly. The cartridge assembly includes a staple cartridge that supports a plurality of staples and defines a first knife slot. The anvil assembly is coupled to the cartridge assembly by a pivot member to facilitate movement of the tool assembly between open and clamped positions. The anvil assembly defines a second knife slot that is aligned with the first knife slot of the cartridge assembly in the clamped position. The tool assembly defines a tissue gap between the cartridge assembly and the anvil assembly when the tool assembly is in the clamped position. The drive assembly includes a drive beam having proximal and distal ends and a working member supported on the distal end of the drive beam. The working member includes an upper beam, a lower beam, and a vertical strut interconnecting the upper beam and the lower beam. The vertical strut includes an upper portion and a lower portion. A first knife has a first cutting edge and is pivotally supported on the upper portion of the vertical strut. A stationary knife is supported on the lower portion of the vertical strut and has a second cutting edge. The first knife is positioned to extend between the cartridge and anvil assemblies such that the first cutting edge of the first knife extends across the tissue gap of the tool assembly when the tool assembly is in the clamped position.

In embodiments, the vertical strut of the working member includes an upper portion and a lower portion, and the first knife is supported on the upper portion of the vertical strut.

In some embodiments, the lower portion of the vertical strut supports a stationary knife having a cutting edge.

In certain embodiments, the lower portion of the vertical strut defines a recess, and the stationary knife is supported within the recess proximally of the distal cutting edge of the first knife.

In embodiments, the distal cutting edge of the first knife is convex and arcuate.

In some embodiments, the distal cutting edge of the first knife is arcuate and convex and the cutting edge of the stationary knife is arcuate and concave.

In certain embodiments, the tool assembly includes an actuation sled having a central rib with an upper surface and cam members, and a proximal portion of the central rib defines a pocket that is positioned to receive the first knife such that the distal cutting edge is positioned above the upper surface of the upper rib.

In embodiments, the pivot member defines an axis and the distal cutting edge of the first knife is positioned to engage tissue below the axis of the pivot member.

In some embodiments, the upper portion of the vertical strut defines a channel and the first knife is pivotally supported within the channel about the pivot member such that the first knife extends distally from the channel.

In embodiments, the first cutting edge is convex and the second cutting edge is concave.

In some embodiments, the first cutting edge is positioned distally of the second cutting edge.

In certain embodiments, the upper portion of the vertical strut defines a channel and the lower portion of the vertical strut defines a recess, wherein the first knife is positioned within the channel and the second knife is positioned within the recess.

Other features of the disclosure will be appreciated from the following description.

### BRIEF DESCRIPTION OF THE DRAWINGS

Various embodiments of the disclosed surgical stapling device are described herein below with reference to the drawings, wherein:

FIG. 1 is a side perspective view of an exemplary embodiment of the disclosed surgical stapling device with a tool assembly of the stapling device in a clamped position;

FIG. 2 is an enlarged view of the indicated area of detail shown in FIG. 1;

FIG. 2A is a cross-sectional view taken along section line 2-2 of FIG. 2;

FIG. 3 is a side perspective view of the a drive assembly of the surgical stapling device shown in FIG. 1;

FIG. 4 is a side perspective view of a working member of the drive assembly shown in FIG. 3 with a knife blade of the working member separated from a body of the working member;

FIG. 5 is a cross-sectional view taken through section line 5-5 of FIG. 3;

FIG. 6 is a cross-sectional view taken through section line 6-6 of FIG. 5;

FIG. 7 is a perspective view from above the distal end of the drive assembly shown in FIG. 3 with the working member engaged with an actuation sled of the tool assembly shown in FIG. 2; and

FIG. 8 is a cross-sectional view taken along section line 8-8 of FIG. 2 illustrating engagement between the knife blade and tissue clamped between the jaws of the tool assembly.

### DETAILED DESCRIPTION OF EMBODIMENTS

The disclosed surgical stapling device will now be described in detail with reference to the drawings in which like reference numerals designate identical or corresponding elements in each of the several views. However, it is to be understood that the disclosed embodiments are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure. In addition, directional terms such as front, rear, upper, lower, top, bottom, distal, proximal, and similar terms are used to assist in understanding the description and are not intended to limit the disclosure.

In this description, the term “proximal” is used generally to refer to that portion of the device that is closer to a clinician, while the term “distal” is used generally to refer to that portion of the device that is farther from the clinician. In addition, the term “endoscopic” is used generally used to refer to endoscopic, laparoscopic, arthroscopic, and/or any other procedure conducted through small diameter incision or cannula. Further, the term “clinician” is used generally to refer to medical personnel including doctors, nurses, and support personnel.

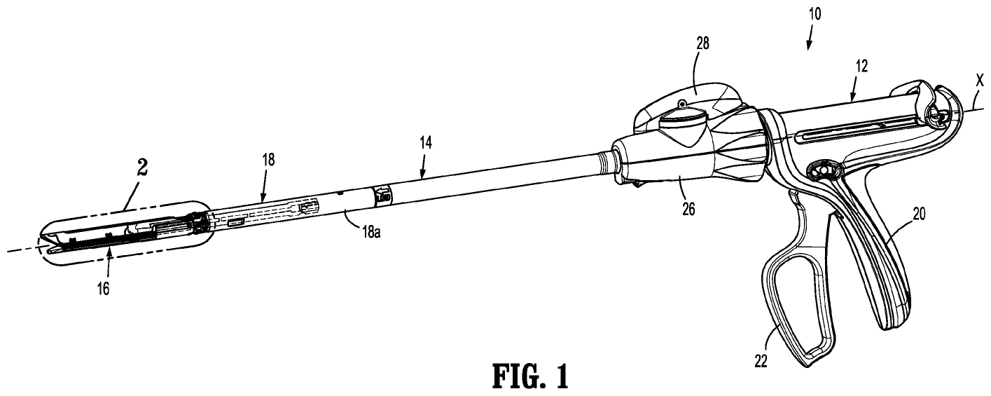

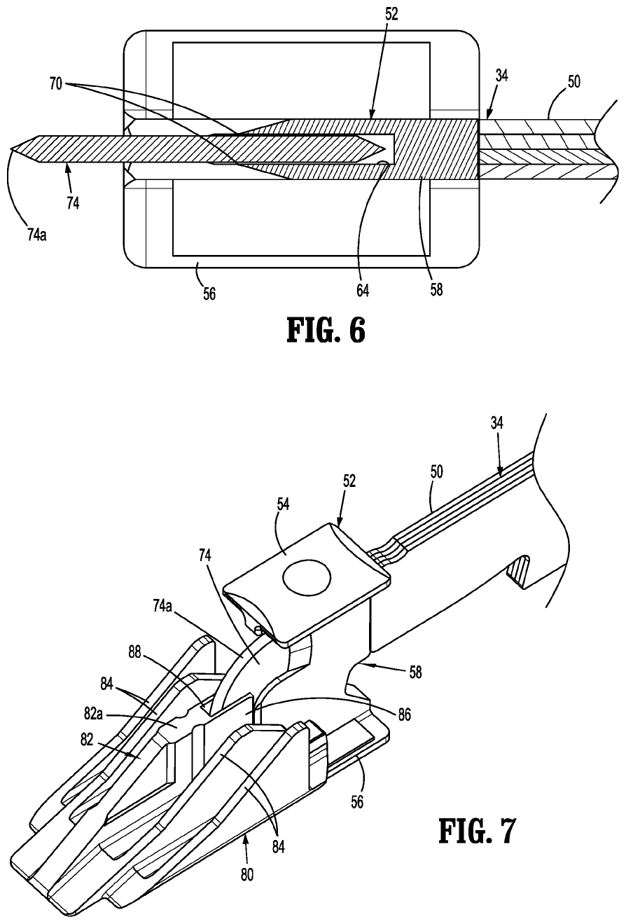

FIGS. 1-8 illustrate an exemplary embodiment of the disclosed surgical stapling device shown as generally as stapling device 10. Stapling device 10 as illustrated in FIG. 1 includes a handle assembly 12, an elongate body 14, and a tool assembly 16. The elongate body 14 defines a longitudinal axis “X” and includes a proximal portion supported on the handle assembly 12 and a distal portion that supports the tool assembly 16. In some embodiments, the tool assembly 16 forms part of a disposable loading unit 18 that includes a proximal body portion 18a. In embodiments, the proximal body portion 18a includes a distal portion that supports the tool assembly 16 and a proximal portion that is adapted to be selectively coupled to and uncoupled from the distal portion of the elongate body 14. In alternate embodiments, the tool assembly 16 is fixedly secured to the distal portion of the elongate body 14.

FIG. 1 illustrates the handle assembly 12 of the stapling device 10 which includes a stationary handle 20 and a firing trigger 22 that is movable in relation to the stationary handle 20 to actuate the tool assembly 16, i.e., approximate and fire staples. As illustrated, the firing trigger 22 is pivotably supported adjacent the stationary handle 20 and is manually movable to actuate the tool assembly 16. It is envisioned that the stapling device 10 can include an electrically powered handle assembly or, alternately, be adapted to be coupled to a robotically controlled system. In some embodiments, the handle assembly 12 supports a rotation knob 26 that supports an articulation lever 28. The rotation knob 26 is supported on a distal portion of the handle assembly 12 and supports the elongate body 14 such that rotation of the rotation knob 26 in relation to the handle assembly 12 causes rotation of the elongate body 14 about the longitudinal axis “X”. The articulation lever 28 is movably supported on the rotation knob 26 to articulate the tool assembly 16 from a position aligned with the longitudinal axis “X” of the elongate body 14 and positions misaligned with the longitudinal axis “X” of the elongate body 14. For a more detailed description of a stapling device including many of the components described above, see, e.g., U.S. Pat. No. 5,865,361 (“the '361 patent”).

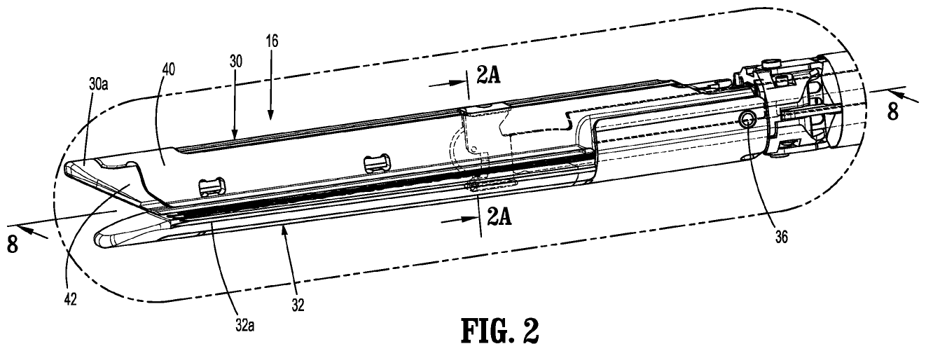

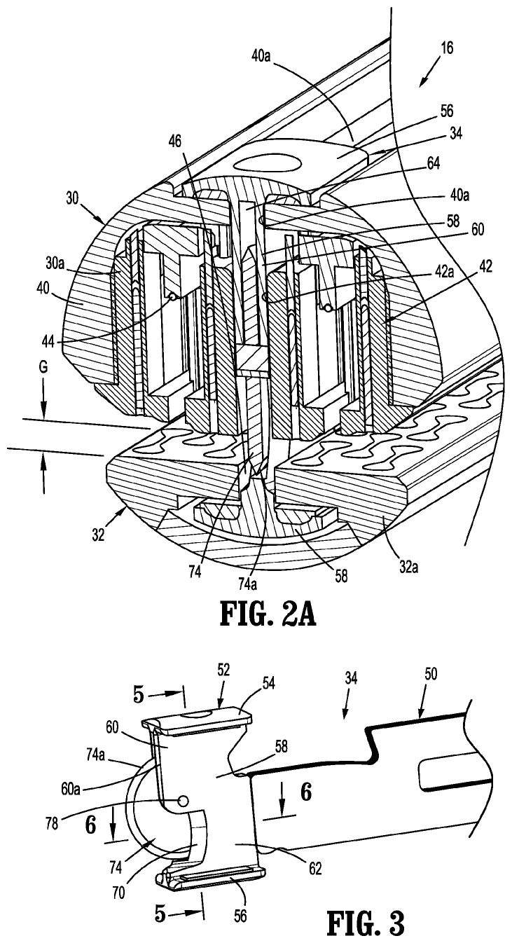

FIGS. 2 and 2A illustrate the tool assembly 16 which includes a first jaw 30 supporting a cartridge assembly 30a, a second jaw 32 supporting an anvil assembly 32a, and a drive assembly 34. The cartridge assembly 30a and the anvil assembly 32a are secured together with a pivot member or pin 36 (FIG. 2) to pivot in relation to each other such that the tool assembly 16 can move between an open position (not shown) and a clamped position (FIG. 2). In the clamped position, the cartridge assembly 30a and the anvil assembly 32a define a tissue gap “G” (FIG. 2A). In embodiments, the cartridge assembly 30a can pivot about the pivot pin 36 in relation to the anvil assembly 32a and the elongate body 14, and the anvil assembly 32a is stationary in relation to the elongate body 14. In other embodiments, the anvil assembly 32a can pivot about the pivot pin 36 in relation to the cartridge assembly 32a and the elongate body 14, and the cartridge assembly 30a is stationary in relation to the elongate body 14.

The cartridge assembly 30a includes a channel 40 and a staple cartridge 42 that is received within the channel 40. In embodiments, the staple cartridge 42 is removable from the channel 40 to allow for replacement of spent or used staple cartridges 42 to facilitate reuse of the stapling device 10. The staple cartridge 42 supports a plurality of staples 44. The channel 40 and the staple cartridge 42 each define a knife slot 40a and 42a (FIG. 2A), respectively. Similarly, the anvil assembly 32a defines a knife slot 46 (FIG. 2A). For a more detailed description of the cartridge assembly 30a and the anvil assembly 32a, see the '361 patent.

FIGS. 2A-6 illustrate a distal portion of the drive assembly 34 which includes a flexible drive beam 50 and a working member 52. The working member 52 includes an upper beam 54, a lower beam 56, and a vertical strut 58 that extends between and supports the upper and lower beams 54, 56. The vertical strut 58 includes an upper portion 60 and a lower portion 62. The upper portion 60 includes a distal surface 60a (FIG. 3) and defines a channel 64 that has a distal end defined by the distal surface 60a. The lower portion 62 defines a lower recess 66 that is positioned beneath the channel 64. The lower recess 66 is defined in part by a proximal wall 68 that is positioned proximally of the distal surface 60a of the upper portion 60. The upper distal surface 60a of the vertical strut 58 defines a distal end of the channel 64.

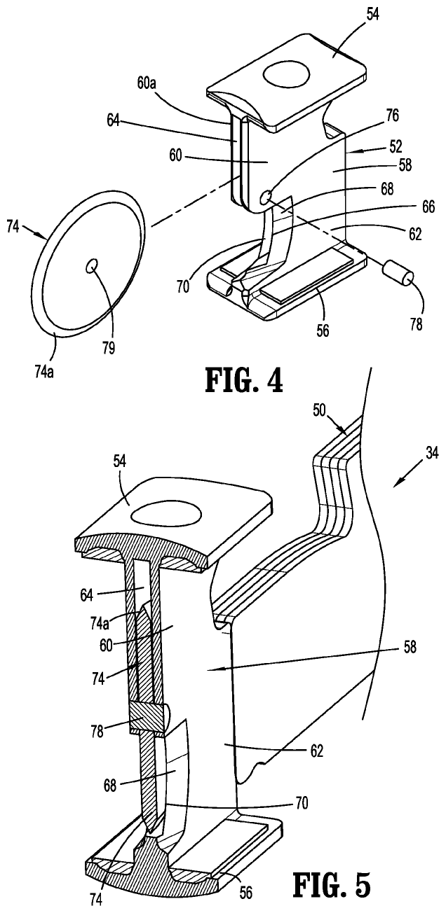

In embodiments, the proximal wall 68 defining the lower recess 66 supports or forms a fixed or stationary knife blade 70 that is positioned proximally of the distal surface 60a of the upper portion 60 of the vertical strut 58 and the channel 64 receives a rotatable knife 74. As used herein, “stationary” means stationary in relation to the vertical strut 68. In some embodiments, the upper portion 60 of the vertical strut 68 defines openings 76 that are positioned on opposite sides of the channel 64 and receive a pivot member or pin 78. The rotatable knife 74 includes a central opening 79 (FIG. 4) that receives the pivot pin 78 to support the rotatable knife 74 for rotation within the channel 64.

The rotatable knife 74 has a curved convex cutting edge 74a that is positioned distally of the distal surface 60a of the upper portion 60 of the vertical strut 58 of the working member 52. In embodiments, the rotatable knife 74 has a circular or saucer-like configuration and the cutting edge 74a extends at least partially about a distal portion of the rotatable knife 74. In other embodiments, the rotatable knife 74 has a distal arcuate or arc-shaped portion that projects distally of the distal surface 60a of the vertical strut 58 and the cutting edge 74 extends at least partially about the distal arc shaped portion of the rotatable knife 74. Although the pivot pin 78 is shown to be coupled to rotatable knife 74 along a central axis of the rotatable knife 74, it is envisioned that the pivot pin 78 may be offset from the central axis of the rotatable knife 74. It is also envisioned that the rotatable knife 74 need not be cylindrical or saucer-shaped but rather can have a variety of different arcuate or wedge shapes in which the cutting edge 74a of the knife 74 is arc-shaped or defined by one or more radii of curvature.

FIG. 7 illustrates the distal end of the drive assembly 34 with the working member 52 of the drive assembly 34 engaged with an actuation sled 80 of the tool assembly 16 (FIG. 2). As known in the medical arts, the actuation sled 80 is engaged and driven by the drive assembly 34 through the tool assembly 16 to eject staples 44 (FIG. 2A) from the cartridge assembly 30a (FIG. 2A). In embodiments, the actuation sled 80 includes a central rib 82 having an upper surface 82a and cam members 84 that are positioned on each side of the central rib 82. A proximal portion 86 of the central rib 82 defines a pocket 88 that receives the rotatable knife 74 when the working member 52 of the drive assembly 34 is engaged with the actuation sled 80. The rotatable knife 74 is received within the pocket 88 of the central rib 82 such that an upper surface of the rotatable knife 74 extends above the upper surface 82a of the central rib 82.

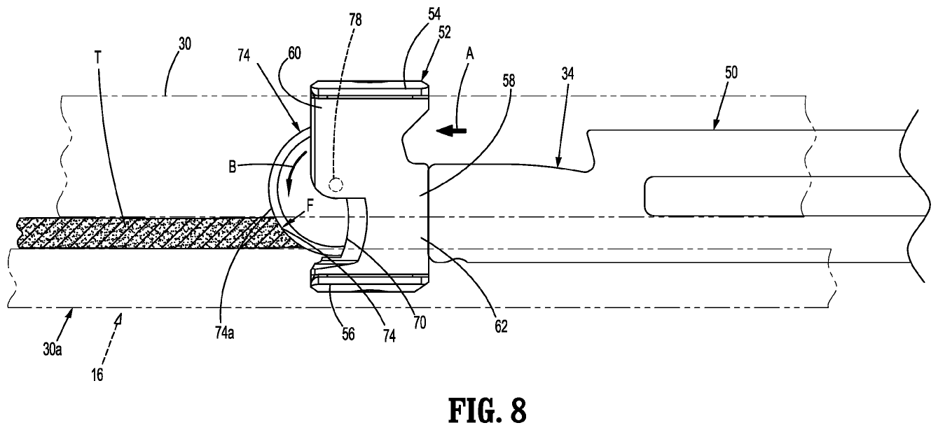

FIG. 8 illustrates the tool assembly 16 clamped about tissue “T” as the working member 52 of the drive assembly 34 is advanced distally in the direction indicated by arrow “A” through the tool assembly 16 to dissect the tissue “T”. When the working member 52 of the drive assembly 34 is positioned within the tool assembly 16, the rotatable knife 74 and the stationary knife 70 extend across the tissue gap “G” (FIG. 2A) between the knife slots 42a and 46 of the staple cartridge 42 and the anvil assembly 32a, respectively, such that the knives 74 and 70 engage the tissue “T” clamped between the cartridge and anvil assemblies 30a, 32a, respectively. As illustrated in FIG. 8, the tissue “T” is positioned to engage the rotatable knife 74 at a position below a horizontal axis that extends through the pivot pin 78 such that the rotatable knife 74 is urged to rotate, upon engagement with the tissue “T”, in the direction indicated by arrow “B” in FIG. 8. The curvature of the rotatable blade 74 applies a force “F” on the tissue “T” that has both a horizontal and vertical component to minimize tissue accumulation and slippage associated with known blades that apply a substantially all horizontal force on the tissue “T”.

The stationary blade 70 is recessed within the working member 52 and positioned proximally of the blade edge 74a of the rotatable blade 74. The stationary blade 70 is positioned to complete cutting of tissue “T” that was not cleanly cut by the rotatable blade 74. In embodiments, the stationary blade 70 has an arcuate, concave shape although other configurations are envisioned.

Although the disclosed working member 52 of the drive assembly 34 of the tool assembly 16 is illustrated to include both a stationary blade 70 and a rotatable blade 74, it is envisioned that the working member may include only one of the blades 70 and 74. For example, the working member 52 may only be provided with a rotatable blade 74.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A tool assembly comprising;
a cartridge assembly including a staple cartridge supporting a plurality of staples, the staple cartridge defining a first knife slot;
an anvil assembly coupled to the cartridge assembly by a pivot member to facilitate movement of the tool assembly between open and clamped positions, the anvil assembly defining a second knife slot that is aligned with the first knife slot of the cartridge assembly in the clamped position of the tool assembly, the tool assembly defining a tissue gap between the cartridge assembly and the anvil assembly when the tool assembly is in the clamped position; and
a drive assembly including a drive beam having proximal and distal ends and a working member supported on the distal end of the drive beam, the working member including an upper beam, a lower beam, and a vertical strut interconnecting the upper beam and the lower beam, and a first knife having a distal cutting edge supported on the vertical strut by a pivot member, the first knife positioned to extend between the cartridge and anvil assemblies such that the distal cutting edge extends across the tissue gap of the tool assembly when the tool assembly is in the clamped position.

2. The tool assembly of claim 1, wherein the vertical strut of the working member includes an upper portion and a lower portion, the first knife supported on the upper portion of the vertical strut.

3. The tool assembly of claim 2, wherein the lower portion of the vertical strut supports a stationary knife having a cutting edge.

4. The tool assembly of claim 3, wherein the lower portion of the vertical strut defines a recess, the stationary knife being supported within the recess proximally of the distal cutting edge of the first knife.

5. The tool assembly of claim 1, wherein the distal cutting edge of the first knife is convex and is arcuate.

6. The tool assembly of claim 3, wherein the distal cutting edge of the first knife is arcuate and convex and the cutting edge of the stationary knife is arcuate and concave.

7. The tool assembly of claim 1, further including an actuation sled, the actuation sled including a central rib having an upper surface and cam members, a proximal portion of the central rib defining a pocket positioned to receive the first knife such that the distal cutting edge is positioned above the upper surface of the upper rib.

8. The tool assembly of claim 1, wherein the pivot member defines an axis and the distal cutting edge of the first knife is positioned to engage tissue below the axis of the pivot member.

9. The tool assembly of claim 2, wherein the upper portion of the vertical strut defines a channel and the first knife is pivotally supported within the channel about the pivot member such that the first knife extends distally from the channel.

10. A tool assembly comprising;
a cartridge assembly including a staple cartridge supporting a plurality of staples, the staple cartridge defining a first knife slot;
an anvil assembly coupled to the cartridge assembly by a pivot member to facilitate movement of the tool assembly between open and clamped positions, the anvil assembly defining a second knife slot that is aligned with the first knife slot of the cartridge assembly in the clamped position of the tool assembly, the tool assembly defining a tissue gap between the cartridge assembly and the anvil assembly when the tool assembly is in the clamped position; and
a drive assembly including a drive beam having proximal and distal ends and a working member supported on the distal end of the drive beam, the working member including an upper beam, a lower beam, and a vertical strut interconnecting the upper beam and the lower beam, the vertical strut including an upper portion and a lower portion, a first knife having a first cutting edge and being pivotally supported on the upper portion of the vertical strut, and a stationary knife supported on the lower portion of the vertical strut having a second cutting edge, the first knife positioned to extend between the cartridge and anvil assemblies such that the first cutting edge of the first knife extends across the tissue gap of the tool assembly when the tool assembly is in the clamped position.

11. The tool assembly of claim 10, wherein the first cutting edge is convex and the second cutting edge is concave.

12. The tool assembly of claim 11, wherein the first cutting edge is positioned distally of the second cutting edge.

13. The tool assembly of claim 12, wherein the upper portion of the vertical strut defines a channel and the lower portion of the vertical strut defines a recess, the first knife positioned within the channel and the second knife positioned within the recess.

14. The tool assembly of claim 10, further including an actuation sled, the actuation sled including a central rib having an upper surface and cam members, a proximal portion of the central rib defining a pocket positioned to receive the first knife such that the first cutting edge is positioned above the upper surface of the upper rib.

15. The tool assembly of claim 10, wherein the first knife is supported on the vertical strut by a pivot member, the pivot member defining an axis and the first cutting edge of the first knife positioned to engage tissue below the axis of the pivot member.

16. A tool assembly comprising;
a cartridge assembly including a staple cartridge supporting a plurality of staples, the staple cartridge defining a first knife slot;
an anvil assembly coupled to the cartridge assembly by a pivot member to facilitate movement of the tool assembly between open and clamped positions, the anvil assembly defining a second knife slot that is aligned with the first knife slot of the cartridge assembly in the clamped position of the tool assembly, the tool assembly defining a tissue gap between the cartridge assembly and the anvil assembly when the tool assembly is in the clamped position; and
a drive assembly including a working member and a first knife, the first knife pivotally supported on the working member and having a first cutting edge, the first knife positioned to extend between the cartridge and anvil assemblies such that the first cutting edge extends across the tissue gap of the tool assembly when the tool assembly is in the clamped position.

17. The tool assembly of claim 16, wherein the distal cutting edge is convex.

18. The tool assembly of claim 17, wherein the working member supports a stationary knife having a second cutting edge.

19. The tool assembly of claim 18, wherein the first cutting edge is positioned distally of the second cutting edge.

20. The tool assembly of claim 19, further including an actuation sled, the actuation sled including a central rib having an upper surface and cam members, a proximal portion of the central rib defining a pocket positioned to receive the first knife such that the first cutting edge is positioned above the upper surface of the upper rib.
